(19) United States
(12) Patent Application Publication
(10) Pub. No.: US 2026/0225979 A1
(43) Pub. Date: Aug. 6, 2026

(54) PROCESS FOR PRODUCING DIALKYL SUCCINATE

(71) Applicant: Johnson Matthey Davy Technologies Limited, London (GB)

(72) Inventors: Henry Arthur CLAXTON, London (GB); Paul GORDON, Stockton-on-Tees (GB); Joshua Andrew HEAPS, Stockton-on-Tees (GB); Graham REED, London (GB); Martin Lucas SMIDT, London (GB)

(21) Appl. No.: 19/150,782

(22) PCT Filed: Feb. 13, 2024
(86) PCT No.: PCT/GB2024/050382

§ 371 Date: Jul. 24, 2025

(30) Foreign Application Priority Data
Feb. 14, 2023 (GB) 2302059.7

(51) Int. Cl. C07C 27/04; C07C 27/28

(52) U.S. Cl. CPC C07C 27/04; C07C 27/28; C07C 2523/755

(57) ABSTRACT

The present invention provides a process for producing dialkyl succinate, the process comprising hydrogenating dialkyl maleate in a multi-stage process comprising at least two hydrogenation stages wherein: in a first hydrogenation stage a feed stream comprising the dialkyl maleate is exposed to hydrogenating conditions over a catalyst at a temperature T to produce a first stream comprising dialkyl succinate and unsaturated compound(s), and in a second hydrogenation stage the first stream is exposed to hydrogenating conditions at a temperature T₂ to produce a second stream comprising dialkyl succinate and a lower concentration of the unsaturated compound(s) than the first stream; wherein T₂ is less than T₁.

### FIELD OF THE INVENTION

The present invention relates to a process for the preparation of dialkyl succinate from dialkyl maleate by a multi-stage hydrogenation.

### BACKGROUND

Dimethyl succinate is widely used in a variety of applications including solvents, and as a chemical intermediate. Recently, there has been growing interest in the use of dimethyl succinate in the production of polybutylene succinate (PBS) by polymerisation with 1,4-butanediol. PBS is coming to the fore as a leading biodegradable plastic.

Various ways of making dimethyl succinate are known, including esterification of succinic acid with methanol using an acid catalyst such as sulfuric acid, and esterification of maleic anhydride to form dimethyl maleate, followed by hydrogenation to form dimethyl succinate. The second process benefits from less side reactions and a more environmentally friendly profile in terms of side-product generation and overall efficiency. The second process also benefits from an ability to integrate with a 1,4-butanediol production process, which can also use dimethyl maleate as a starting material. The dimethyl maleate is exposed to hydrogenation and hydrogenolysis and proceeds via dimethyl succinate to produce 1,4-butanediol, along with tetrahydrofuran and γ-butyrolactone which also have commercial value.

Processes for the esterification of maleic anhydride to form dimethyl maleate are disclosed, for example, in U.S. Pat. No. 4,795,824 and WO 90/08127. Dimethyl maleate can be hydrogenated to form dimethyl succinate by various means, typically using molecular hydrogen and a platinum group metal catalyst, e.g. palladium supported on carbon. The reaction is highly exothermic, and it can be beneficial to operate the hydrogenation reactor at higher temperatures to recover the energy as useful heat which allows steam to be raised for use elsewhere on a plant. Operating the reactor at high temperature increases the reaction kinetics, reducing catalyst bed size.

The present inventors have found that the downside of operating at high temperatures with platinum group metal catalysts such as palladium supported on carbon is that relatively high levels of γ-butyrolactone can be formed when the reaction is operated at high conversions. The reaction would be operated at high conversions to minimise the concentration of unconverted unsaturated compounds, i.e. dimethyl maleate and dimethyl fumarate.

Hence, it is currently not viable to achieve low levels of unsaturated compounds along with γ-butyrolactone whilst also operating at temperatures high enough to produce useful heat which can be used elsewhere in the plant. Moreover, the present inventors have found that dimethyl maleate, dimethyl fumarate and in particular γ-butyrolactone cannot be readily separated from dimethyl succinate e.g. cannot be separated without high energy input and multiple distillation columns. Both the unsaturated compounds and γ-butyrolactone in the dimethyl succinate product impact on the quality of PBS formed from the polymerisation of the dimethyl succinate with 1,4-butanediol.

### SUMMARY OF THE INVENTION

Accordingly, the present invention provides a process for producing dialkyl succinate, the process comprising hydrogenating dialkyl maleate in a multi-stage process comprising at least two hydrogenation stages wherein:
in a first hydrogenation stage a feed stream comprising the dialkyl maleate is exposed to hydrogenating conditions over a catalyst at a temperature T₁ to produce a first stream comprising dialkyl succinate and unsaturated compound(s), and
in a second hydrogenation stage the first stream is exposed to hydrogenating conditions at a temperature T₂ to produce a second stream comprising dialkyl succinate and a lower concentration of the unsaturated compound(s) than the first stream;
wherein T₂ is less than T₁.

The unsaturated compound(s) is/are dialkyl maleate and optionally dialkyl fumarate, typically dialkyl maleate and dialkyl fumarate. The dialkyl maleate is unconverted from the feed stream. Dialkyl fumarate typically arises from isomerisation of the dialkyl maleate.

The inventors have advantageously found that the process enables the preparation of dialkyl succinate with minimal γ-butyrolactone, such that a very high purity dialkyl succinate product can be provided after refining by distillation. This is possible whilst also maximising heat recovery from the process, which reduces plant operating costs and environmental impact. By optionally utilising a base metal catalyst in the second hydrogenation stage, further benefits can be seen in that the second hydrogenation stage can be made more active at the lower temperature without affecting product purity. So, it is additionally possible to minimise reactor size and catalyst volume which has further benefits in terms of operating costs and environmental impact.

Also provided is a process for producing polybutylene succinate, the process comprising producing dialkyl succinate by the process of the present disclosure, then polymerising the dialkyl succinate with 1,4-butanediol in a subsequent polymerisation stage.

### BRIEF DESCRIPTION OF THE DRAWINGS

FIG. 1 is a schematic diagram of the experimental set-up used in the examples.

FIG. 2 is a schematic diagram of a process of the invention.

FIG. 3 is a chart showing γ-butyrolactone formation vs overall conversion of unsaturated compounds at various temperatures.

### DETAILED DESCRIPTION OF THE INVENTION

The dialkyl maleate may be a C₁ to C₅ dialkyl maleate, e.g. dimethyl, diethyl, dipropyl, dibutyl or dipentyl maleate. Preferably, the dialkyl maleate is dimethyl or diethyl maleate, more preferably dimethyl maleate.

Accordingly, alkyl used herein may be methyl, ethyl, propyl, butyl or pentyl, preferably methyl or ethyl, more preferably methyl.

The feed stream for the first hydrogenation stage comprising dialkyl maleate may be obtained by esterification of maleic anhydride with the corresponding alkyl alcohol. Suitably, the esterification is a two-stage esterification in which maleic anhydride or maleic acid is esterified to produce monoalkyl maleate in a first stage, and the monoalkyl maleate is esterified to produce dialkyl maleate in a second stage. The second stage may be a reactive distillation using an acidic resin catalyst, preferably a sulphonic acid-based ion-exchange resin. The feed stream comprising dialkyl maleate preferably comprises at least about 90 wt % dialkyl maleate, preferably at least about 95 wt %. The feed stream comprising dialkyl maleate preferably comprises less than about 1 wt % monoalkyl maleate, preferably less than about 0.5 wt % monoalkyl maleate. The feed stream comprising dialkyl maleate preferably comprises less than or equal to about 5 wt % alkyl alcohol. Suitable processes for the esterification of maleic anhydride to form dialkyl maleate are disclosed, for example, in U.S. Pat. No. 4,795,824 and WO 90/08127 which are incorporated herein by reference.

Suitably, the feed stream comprising dialkyl maleate is treated to reduce levels of sulphur to less than about 0.2 ppmw, preferably by passing the stream over a guard bed. The feed stream comprising dialkyl maleate may be treated to neutralise any monoalkyl maleate present, preferably by treatment with a base selected from NaOH, Na₂CO₃, NaHCO₃ or an alkyl amine.

Typically, the first hydrogenation stage is carried out in a first hydrogenation reactor and the second hydrogenation stage is carried out in a second hydrogenation reactor. As will be evident to a skilled person, such reactors are operated in series. However, the two stages may be carried out in a single reactor, for example having two or more zones. Preferably, there are two hydrogenation stages. The first and second hydrogenation stages may typically be liquid or mixed vapour/liquid phase reaction stages. Put another way the conditions are such as to maintain a liquid or mixed liquid/vapour stage. That may be achieved, for example, by controlling one or more conditions such as feed ratio of hydrogen to the feed stream comprising dialkyl maleate, the pressure and the temperature. The first hydrogenation stage may be carried out in a trickle bed reactor, but the type of reactor is not particularly limited and any reactor suitable for the desired reaction may be used.

A source of hydrogen, typically hydrogen gas, will be added to the first hydrogenation stage. Hydrogen gas is preferably fed into the stage in molar excess with respect to the dialkyl maleate. The hydrogen partial pressure in the first hydrogenation stage is typically in the range of and including about 5 to about 150 barg, preferably about 20 to about 100 about barg, for example about 60 barg.

In the first hydrogenation stage, dialkyl maleate is hydrogenated over a catalyst to provide dialkyl succinate, together with unreacted dialkyl maleate and optionally its isomer dialkyl fumarate, optionally together with γ-butyrolactone. T₁ is suitably at least about 140° C., preferably at least about 150° C. At lower temperatures, the reaction becomes less practical to run and recover useful heat. T₁ is suitably no more than about 220° C., preferably no more than about 200° C. At higher temperatures, the make of unwanted by-products can become too high which can lead to thermal runaway. Running the first hydrogenation stage at such a temperature T₁ means that useful heat can be extracted, e.g. low-pressure steam can be raised and used as a heat transfer fluid elsewhere on the plant, negating the need for additional means on the plant to raise steam and so conserving energy on the plant. Suitably, the catalyst in the first hydrogenation stage is in a catalyst bed, and the temperature T₁ is the temperature at the inlet of the catalyst bed. A skilled person will understand how to control the temperature T₁, including, for example, by controlling the amount and temperature of a liquid recycle. The inventors have found that running the first hydrogenation stage at a temperature T₁ results in the formation of γ-butyrolactone, with the amount of γ-butyrolactone made at a certain temperature T₁ being dependent on dialkyl maleate conversion. The lower T₁, the higher dialkyl maleate conversion may be before γ-butyrolactone becomes impractical. The higher T₁, the lower dialkyl maleate conversion may be before γ-butyrolactone becomes impractical. As will be understood by a skilled person, conversion can be controlled by factors such as catalyst loading (i.e. the amount of catalyst per unit of fresh feed), temperature, pressure and the rate of a liquid recycle. For example, the first hydrogenation stage may be operated such that the dialkyl maleate conversion is 99.95% or less. Typically, the first hydrogenation stage is operated such that the dialkyl maleate conversion is at least about 25%, preferably at least about 50%, more preferably at least about 75%.

The amount of unsaturated compound(s), i.e. total dialkyl maleate and dialkyl fumarate, will depend on the conversion of dialkyl maleate operated in the first hydrogenation stage. The first stream typically comprises less than about 1000 ppmw, preferably less than about 250 ppmw, more preferably less than about 100 ppmw of γ-butyrolactone. The first stream may be substantially free of γ-butyrolactone. This may of course require a relatively low dialkyl maleate conversion in the first hydrogenation stage.

The catalyst in the first hydrogenation stage comprises an active metal for hydrogenating dialkyl maleate to dialkyl succinate. The active metal may comprise a platinum group metal, in particular palladium, platinum, rhodium or ruthenium. Preferably, the catalyst comprises a support, i.e. the catalyst is a supported catalyst. Suitably, the support comprises alumina, silica, zirconia, zinc oxide, chromate, carbon or mixtures thereof. The liquid hourly space velocity in the first hydrogenation stage is typically at most about 50 h⁻¹ based on dialkyl maleate in the feed stream to the first hydrogenation stage. A skilled person can determine the liquid hour space velocity required for a particular system to achieve a desired conversion of dialkyl maleate.

The first hydrogenation stage typically includes a liquid recycle in which the dialkyl maleate feed is diluted with dialkyl succinate from the first stream. The liquid recycle is typically operated such that molar ratio of dialkyl maleate to dialkyl succinate fed into the hydrogenation stage is maintained in the range of 10:1 to 100:1, preferably 20:1 to 30:1. Such a liquid recycle can help to modulate temperature in the first hydrogenation stage. Typically, the liquid recycle will proceed via a heat exchanger to reduce the temperature, and optionally a pump. This heat exchanger can be utilised to provide heat to another stream in the process or the plant in which the process operates, or to raise steam, e.g. low-pressure steam can be raised and used as a heat transfer fluid elsewhere on the plant, negating the need for additional means on the plant to raise steam and so conserving energy on the plant.

In the second hydrogenation stage, which can be considered a polishing stage, unconverted dialkyl maleate, along with any dialkyl fumarate, in the first stream is hydrogenated over a catalyst to provide dialkyl succinate. T₂ is suitably at least about 40° C., preferably at least about 50° C. T₂ is suitably less than about 140° C., preferably no more than about 120° C. As required, the first stream may be cooled to a temperature T₂ by in a cooling stage, for example using cooling water or by heat exchange with a stream from another part of the process or plant in which the process operates. Advantageously, operating at a temperature T₂ results in the second stage completing the hydrogenation of the unsaturated compound(s) to dialkyl succinate, without increasing the amount of γ-butyrolactone with respect to the first stream. As will be evident to a skilled person, 1,4-butanediol, which is produced via the hydrogenolysis of γ-butyrolactone, is typically not produced at a detectable level in the second hydrogenation stage. As such the process maximises the yield of dialkyl succinate. Therefore, impurity levels are low enough in the second stream to allow effective refining by distillation to provide very high purity dialkyl succinate. Accordingly, T₂ can be defined as a temperature at which γ-butyrolactone is not produced under the hydrogenation conditions in the second hydrogenation stage.

The second stream, i.e. the product stream from the second hydrogenation stage typically comprises less than about 1000 ppmw, preferably less than about 250 ppmw, more preferably less than about 100 ppmw of unsaturated compound(s), i.e. total dialkyl maleate and dialkyl fumarate. The second stream typically comprises less than about 1000 ppmw, preferably less than about 250 ppmw, more preferably less than about 100 ppmw of γ-butyrolactone. Other minor impurities in the second stream which are not unsaturated compound(s) or γ-butyrolactone, such as alkyl alcohol, monoalkyl succinate, and dialkyl-2-alkoxy succinate (derived from the reaction of alkyl alcohol with an unsaturated compound) will substantially depend on the composition of the feed to the first hydrogenation zone which comprises the dialkyl maleate. Such impurities will pass through the hydrogenation stages but can be readily removed by distillation. The second stream is a dialkyl succinate-rich stream comprising predominantly dialkyl succinate, for example greater than about 80 wt %.

The second stage is suitably operated such that conversion of the unsaturated compound(s) in the first stream, is maximised, i.e. it is greater than about 99%, preferably greater than 99.99%. As will be understood by a skilled person, conversion can be controlled by factors such as catalyst loading (i.e. the amount of catalyst per unit of fresh feed), temperature, pressure and the rate of a liquid recycle.

A source of hydrogen, typically hydrogen gas, will be added to the second hydrogenation stage. Hydrogen gas is preferably fed into the stage in molar excess with respect to the dialkyl maleate. The hydrogen partial pressure in the second hydrogenation stage is typically in the range of and including about 5 to about 150 barg, preferably about 20 to 100 about barg, for example about 60 barg.

The catalyst in the second hydrogenation stage comprises an active metal for hydrogenating dialkyl maleate to dialkyl succinate. The active metal may comprise a platinum group metal, in particular palladium, platinum, rhodium or ruthenium or a base metal. Advantageously, the active metal comprises a base metal, preferable nickel or copper, more preferably nickel. In this case, further benefits can be seen in that the second hydrogenation stage can be made more active at the lower temperature without affecting product purity. So, it is additionally possible to minimise reactor size and catalyst volume which has further benefits in terms of operating costs and environmental impact. Preferably, the catalyst comprises a support, i.e. the catalyst is a supported catalyst. Suitably, the support comprises alumina, silica, zirconia, zinc oxide, chromate, carbon or mixtures thereof. A skilled person can determine the liquid hour space velocity required for a particular system to achieve maximal conversion of the unsaturated compound(s).

The second hydrogenation stage may include a liquid recycle in which the first stream is diluted with dialkyl succinate from the second stream. Typically, the liquid recycle will proceed via a pump and then a heat exchanger, e.g. utilising a cooling medium such as air or water, to reduce the temperature.

The second stream is typically fed to a refining zone where dialkyl succinate can advantageously be provided with very high purity, in particular having very low levels of γ-butyrolactone. The refining zone suitably comprises a distillation column operated to produce a dialkyl succinate stream comprising greater than about 99.5 wt % dialkyl succinate. A skilled person can determine what packing to use and, for example, how many trays are required. Moreover, a skilled person can determine the operating conditions required in terms of, for example, pressure, temperature and residence time. The high purity of dialkyl succinate which can be produced does not derive from particulars of the refining zone, but rather derives from the low concentration of γ-butyrolactone in the feed to the refining zone, which is an advantageous effect of the invention, as is not practical to separate the γ-butyrolactone by distilling the product due to the low relative volatility of γ-butyrolactone. Suitably, the distillation column is operated to produce an overhead stream comprising alkyl alcohol, i.e. alkyl alcohol, which may be present in the initial feed stream to the first hydrogenation zone, and a bottom stream comprising heavies.

The dialkyl succinate produced by the present process may be used for any means, in particular for producing polybutylene succinate. Accordingly, provided herein is a process for producing polybutylene succinate, the process comprising producing dialkyl succinate by the process disclosed herein, then polymerising the dialkyl succinate with 1,4-butanediol in a subsequent polymerisation stage. Suitably, the 1,4-butanediol is produced from dialkyl maleate i.e. by hydrogenation and hydrogenolysis. Advantageously, the dialkyl succinate, 1,4-butanediol, and polybutylene succinate are produced on the same plant.

The present invention will now be described, by way of example, with reference to the accompanying figures. It will be understood by those skilled in the art that the drawings are diagrammatic and that further items of equipment such as reflux drums, pumps, vacuum pumps, compressors, gas recycle compressors, temperature sensors, pressure relief valves, control valves, flow controllers, level controllers, and the like may be required in a commercial plant. The provision of such ancillary items of equipment forms no part of the present invention and is in accordance with conventional chemical engineering practice.

In FIG. 2 a feed 1 of dialkyl maleate from a maleic anhydride esterification stage (not shown) is supplied to a guard bed 3 to reduce levels of sulphur to less than about 0.2 ppmw. A feed stream 5 from the guard bed is then supplied to a first hydrogenation stage comprising hydrogenation reactor 7. The feed stream contains less than 0.5 wt % monoalkyl maleate, less than 5 wt % alkyl alcohol (carried through from the esterification reaction) and less than 2 wt % dialkyl fumarate. Hydrogenation reactor 7 is a trickle bed reactor housing a catalyst bed which contains a palladium on carbon hydrogenation catalyst. Hydrogen gas is fed into reactor 7 via line 9 in molar excess with respect to dialkyl maleate. Reactor 7 is operated such that the temperature at the inlet of the catalyst bed is T₁ and the dialkyl maleate conversion is such than an acceptable amount of unsaturated compound(s) as well as γ-butyrolactone are present, for example less than 100 ppmw unsaturated compound(s) and less than 100 ppmw γ-butyrolactone. The reactor provides a product stream 11 which contains predominantly the desired reduction product dialkyl succinate along with the unsaturated compound(s) and γ-butyrolactone and any impurities, such as alkyl alcohol present in feed stream 5. The first hydrogenation stage comprises a liquid recycle 17 in which the dialkyl maleate feed 5 is diluted with dialkyl succinate from the product stream 11 from reactor 7. The liquid recycle is operated such that molar ratio of dialkyl maleate to dialkyl succinate in the feed is maintained at about 20:1. The liquid recycle helps to modulate temperature in reactor 7. Moreover, because reactor 7 is operated at a temperature T₁, useful heat can be extracted via heat exchanger 25 and used to raise steam for use elsewhere in the plant. Product stream 11 from reactor 7 is used as a feed stream for hydrogenation reactor 13 in a second hydrogenation stage. Hydrogenation reactor 13 is a trickle bed reactor housing a catalyst bed which contains a catalyst composed of nickel on an alumina-based support. Hydrogen gas is fed into reactor 13 via line 15 in molar excess with respect to dialkyl maleate. Reactor 13 is operated such that the temperature at the inlet of the catalyst bed is T₂. The conversion of unsaturated compound(s) is greater than 99.99% in this reactor. Accordingly, reactor 13 can be considered a polishing reactor which reduced unsaturated compound(s) and does not increase the concentration of γ-butyrolactone. Product stream 17 from reactor 13 comprises predominantly dialkyl succinate with less than 100 ppmw unsaturated compound(s) and less than 100 ppmw γ-butyrolactone. The second hydrogenation stage optionally comprises a liquid recycle 19 in which the dialkyl maleate feed 11 is diluted with dialkyl succinate from the crude product stream 17 from reactor 13. The liquid recycle helps to modulate temperature in reactor 13. The recycle is typically cooled using water in a heat exchanger 23. Product stream 17 is fed to a refining zone 21 which produces dialkyl succinate refined product stream 27 comprising greater than 99.5 wt % dialkyl succinate. Refining zone 21 contains at least one distillation column which is operated using common general knowledge. The extremely high purity of the dialkyl succinate in product stream 27 is only possible because of the low content of γ-butyrolactone in crude product stream 17.

### EXAMPLES

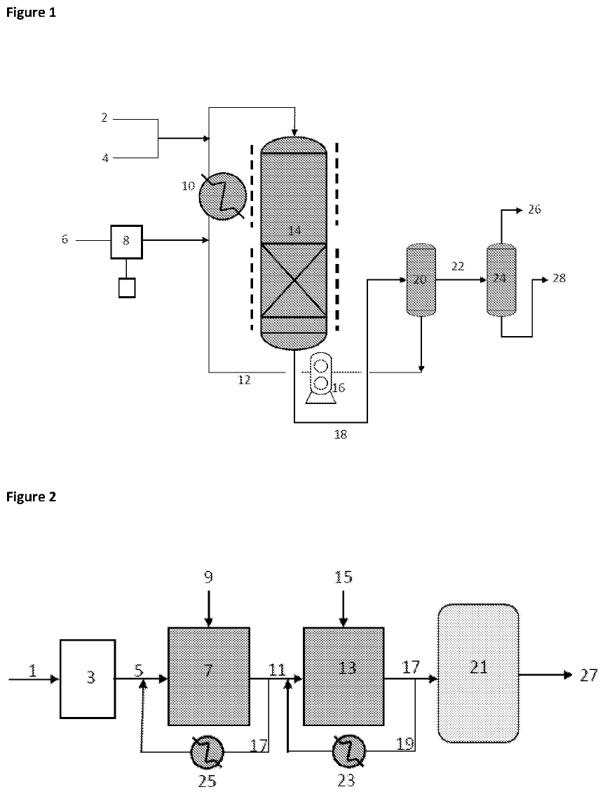

To a trickle bed liquid recycle pilot plant unit as shown in FIG. 1 was charged a 1.5 wt % Pd on C hydrogenation catalyst (50 mL).

In FIG. 1, 2 is a hydrogen feed, 4 is a nitrogen feed, 6 is a dimethyl maleate feed, 8 is feed pump, 10 is a heat exchanger to take heat from liquid recycle 12, 14 is a trickle bed reactor containing the required catalyst, 16 is a gear pump, 18 is a crude product stream, 20 is high pressure liquid recycle vessel, 22 is a crude product stream, 24 is high pressure product let down vessel, 26 is exit gas and 28 is degassed crude dimethyl succinate product.

The catalyst was activated by heating under a continuous flow of hydrogen before the introduction of dimethyl succinate feed via a reciprocating pump. Once dimethyl succinate product was observed a liquid recycle was started (20:1 wt:wt) and the feed was changed to a process stream containing mostly dimethyl maleate (Table 1).

TABLE 1
Process Feed Composition

| Component | Concentration, wt % |
|---|---|
| methanol | 4.40 |
| γ-butyrolactone | 0.00 |
| dimethyl maleate | 92.22 |
| dimethyl fumarate | 1.84 |
| dimethyl-2-methoxy succinate | 0.79 |
| monomethyl maleate | 0.44 |
| Water | 0.10 |
| Others | 0.21 |

Feed and product samples were analysed by GC-FID to determine the concentration of unsaturated compounds (dimethyl maleate+dimethyl fumarate) and γ-butyrolactone in the crude product. Tests were performed at a constant temperature (140° C. inlet), pressure (880 psig) and recycle (20:1). The liquid feed rate and hence liquid hourly space velocity (LHSV, calculated as Feed Rate (mL h⁻¹)/catalyst volume (mL)) was adjusted as required.

Table 2 shows the results from operation at 140° C. reactor temperature, where conversion is either a factor of catalyst deactivation or changes in LHSV (determined as Feed Rate (mL h⁻¹)/Catalyst Volume (ml)).

TABLE 2
Test data at 140° C. reactor inlet temperature

| Run | 1 | 2 | 3 |
|---|---|---|---|
| Operating Conditions | | | |
| Reactor Inlet Temp, ° C. | 140 | 140 | 140 |
| Reactor Pressure, psig | 880 | 880 | 880 |
| LHSV, h⁻¹ | 2.6 | 2.6 | 3.1 |
| Recycle Ratio, wt:wt | 20 | 20 | 20 |
| Product Analysis | | | |
| Total Unsats. (DMM + DMF), ppmwt | 695 | 1087 | 1881 |
| γ-butyrolactone, ppmwt | 455 | 279 | 171 |
| Overall Conversion, wt % | 99.93 | 99.88 | 99.80 |

DMM = dimethyl maleate,
DMF = dimethyl fumarate

### Impact of Higher Temperature (215° C. Inlet) on γ-Butyrolactone Formation

### Experiment 1

To a trickle bed liquid recycle pilot plant unit (FIG. 1) was charged a Pd on C catalyst.

The catalyst was activated by heating under a continuous flow of hydrogen before the introduction of dimethyl succinate feed via a reciprocating pump. Once product was observed the liquid recycle was started (20:1 wt:wt) and the feed was changed to a process stream as described in Table 1.

The unit was operated under conditions shown in Table 3.

TABLE 3
High temperature operating conditions

| Parameter | Target |
|---|---|
| Reactor Inlet Temperature, ° C. | 215 |
| Reactor Pressure, psig | 880 |
| LHSV, h⁻¹ | 3.0 |
| Recycle Ratio, wt:wt | 20:1 |

The unit was operated under these conditions for 220 hours, during which time the overall conversion stabilised at approximately 99.37 wt %, with key components at the follow levels: dimethyl maleate 4390 ppmwt, dimethyl fumarate 1536 ppmwt and γ-butyrolactone 4161 ppmwt.

### Experiment 2

Experiment 1 was repeated but with aged catalyst to lower conversion. Once stable this afforded an overall conversion of 97.93 wt %, with key components at the follow levels: dimethyl maleate 13768 ppmw), dimethyl fumarate 5669 ppmwt and γ-butyrolactone 550 ppmwt.

### Experiment 3

Experiment 1 was repeated with further aged catalyst, which had undergone a high temperature regeneration step. Once stable this afforded an overall conversion of 99.28 wt %, with key components at the follow levels dimethyl maleate: 5117 ppmwt, dimethyl fumarate 1694 ppmwt and γ-butyrolactone 1674 ppmwt.

Demonstrating Low γ-Butyrolactone Formation/High Conversion when Operating at Low Temperature-Ni Catalyst

To a trickle bed reactor as described in Example 1 was charged a Ni on Al₂O₃/SiO₂ catalyst (62.5 mL).

The catalyst was activated by heating under a continuous flow of hydrogen before the introduction of dimethyl succinate feed via a reciprocating pump. Once product was observed the liquid recycle was started (20:1 wt:wt) and the feed was changed to the feed shown in Table 1.

The Liquid Recycle unit was operated under the conditions shown in Table 4 using the same feed shown in Table 1.

TABLE 4
Low temperature operating conditions

| Parameter | Target |
|---|---|
| Reactor Inlet Temperature, ° C. | 75 |
| Reactor Pressure, psig | 880 |
| LHSV, h⁻¹ | 3.5 |
| Recycle Ratio, wt:wt | 20:1 |

The unit was operated under these conditions for 49 hours, with dimethyl maleate stabilising at 41 ppmwt in the crude product. No dimethyl fumarate or γ-butyrolactone were observed.

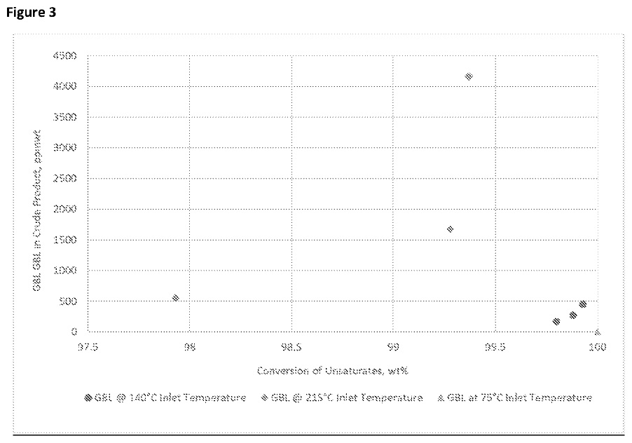

FIG. 3 shows a comparison of γ-butyrolactone formation vs overall conversion of unsaturated compounds at 75° C. (Ni catalyst), 140° C. and 215° C. (Pd catalyst).

### Demonstrating Second Hydrogenation Stage—Polisher Reactor

The unit was operated under the conditions shown in Table 5 using the Ni on Al₂O₃/SiO₂ catalyst. The feed for this run was crude product from previous runs, as shown in Table 6, such that the reactor is operating as a second hydrogenation stage in accordance with the invention.

TABLE 5
Second reactor operating conditions

| Parameter | Target |
|---|---|
| Reactor Inlet Temperature, ° C. | 85 |
| Reactor Pressure, psig | 880 |
| LHSV, h⁻¹ | 3.5 |
| Recycle Ratio, wt:wt | 20:1 |

TABLE 6
Feed to polisher reactor

| Component | Concentration, wt % |
|---|---|
| methanol | 5.982 |
| γ-butyrolactone | 0.000 |
| dimethyl maleate | 0.366 |
| dimethyl fumarate | 0.013 |
| dimethyl succinate | 89.594 |
| dimethyl-2-methoxy succinate | 0.835 |
| monomethyl succinate | 0.400 |
| Water | 0.540 |
| Others | 2.70 |

The unit was operated under these conditions for 218 hours during which time dimethyl maleate in the crude product stabilised at 15 to 22 ppmwt (>99.99 wt % conversion), with no evidence of any dimethyl fumarate or γ-butyrolactone in the crude product. Accordingly, unsaturated compounds were converted and no γ-butyrolactone was made.

These data demonstrate that at temperatures of 140° C. and above and when the hydrogenation is operated at high conversion of dialkyl maleate, γ-butyrolactone make increases to levels which can create problems for downstream refining (e.g. as shown in FIG. 3). Accordingly, when operating at such temperatures to facilitate recovery of the energy as useful heat, γ-butyrolactone make is sub-optimal. So, the hydrogenation must be run at lower conversions for example conversions less than 99.9%. This itself is not optimal, but the problem can be remedied by using a second hydrogenation stage according to the invention. Such a second hydrogenation stage acts as a polishing stage and enable >99.99 wt % conversion of remaining unsaturated compounds with no additional γ-butyrolactone make. In the particular case of a nickel catalyst the polishing hydrogenation can be made more active at the lower temperature without affecting product purity.

1. A process for producing dialkyl succinate, the process comprising hydrogenating dialkyl maleate in a multi-stage process comprising at least two hydrogenation stages wherein:
in a first hydrogenation stage a feed stream comprising the dialkyl maleate is exposed to hydrogenating conditions over a catalyst at a temperature T₁ to produce a first stream comprising dialkyl succinate and unsaturated compound(s), and
in a second hydrogenation stage the first stream is exposed to hydrogenating conditions at a temperature T₂ to produce a second stream comprising dialkyl succinate and a lower concentration of the unsaturated compound(s) than the first stream;
wherein T₂ is less than T₁.

2. The process according to claim 1, wherein T₁ is at least about 140° C.

3. The process according to claim 1, wherein T₂ is less than about 140° C.

4. The process according to claim 1, wherein in the first hydrogenation stage the feed stream is exposed to hydrogenating conditions over a platinum group metal catalyst, which is optionally a supported catalyst.

5. The process according to claim 1, wherein in the second hydrogenation stage the first stream is exposed to hydrogenating conditions over a platinum group metal catalyst, which is optionally a supported catalyst.

6. The process according to claim 1, wherein in the second hydrogenation stage the first stream is exposed to hydrogenating conditions over a base metal catalyst, which is optionally a supported catalyst.

7. The process according to claim 6, wherein the base metal is nickel.

8. The process according to claim 1, wherein the first hydrogenation stage includes a liquid recycle in which the dialkyl maleate feed is diluted with dialkyl succinate from the first stream.

9. The process according to claim 8, wherein the liquid recycle is operated such that the molar ratio of dialkyl maleate to dialkyl succinate fed into the hydrogenation stage is maintained in the range of 10:1 to 100:1, preferably 20:1 to 30:1.

10. The process according to claim 1, wherein the first stream comprises less than about 1000 ppmw, preferably less than about 250 ppmw, more preferably less than about 100 ppmw of γ-butyrolactone.

11. The process according to claim 1, wherein the second stream comprises less than about 1000 ppmw, preferably less than about 250 ppmw, more preferably less than about 100 ppmw of the unsaturated compound(s).

12. The process according to claim 1, wherein the second stream comprises less than about 1000 ppmw, preferably less than about 250 ppmw, more preferably less than about 100 ppmw of γ-butyrolactone.

13. The process according to claim 1, wherein the second stream is fed to a refining zone.

14. The process according to claim 13, wherein the refining zone comprises a distillation column operated to produce a dialkyl succinate stream comprising greater than about 99.5 wt % dialkyl succinate.

15. The process according to claim 1, wherein the feed stream comprising dialkyl maleate is obtained by esterification of maleic anhydride with the corresponding alkyl alcohol.

16. The process according to claim 15, wherein the feed stream comprising dialkyl maleate comprises less than about 1 wt % monoalkyl maleate, preferably less than about 0.5 wt % monoalkyl maleate.

17. The process according to claim 15, wherein the feed stream comprising dialkyl maleate comprises less than or equal to about 5 wt % alkyl alcohol.

18. The process for producing polybutylene succinate, the process comprising producing dialkyl succinate by the process of claim 1, then polymerising the dialkyl succinate with 1,4-butanediol in a subsequent polymerisation stage.

19. The process according to claim 18, wherein the 1,4-butanediol is produced from a dialkyl maleate.

20. The process according to claim 18, wherein the dialkyl succinate, 1,4-butanediol, and polybutylene succinate are produced on the same plant.

21. The process according to claim 1, wherein alkyl is methyl.
